(19) United States
(12) Patent Application Publication
(10) Pub. No.: US 2005/0187514 A1
(43) Pub. Date: Aug. 25, 2005

(54) Apparatus and method for an ultrasonic medical device operating in a torsional mode

(75) Inventors: Robert A. Rabiner, North Reading, MA (US); Bradley A. Hare, Chelmsford, MA (US); Rebecca I. Marciante, North Reading, MA (US); Mark J. Varady, Andover, MA (US)

Correspondence Address: FISH & RICHARDSON PC, 225 FRANKLIN ST, BOSTON, MA, 02110, US

(73) Assignee: OmniSonics Medical Technologies, Inc.

(21) Appl. No.: 10/774,985

(22) Filed: Feb. 9, 2004

(51) Int. Cl. A61B 17/20

(52) U.S. Cl. 604/22

(57) ABSTRACT

The present invention provides an apparatus and a method for an ultrasonic medical device operating in a torsional mode to treat a biological material. An ultrasonic probe of the ultrasonic medical device is placed in communication with the biological material. An ultrasonic energy source is activated to produce an electrical signal that drives a transducer to produce a torsional vibration of the ultrasonic probe. The torsional vibration produces a rotation and a counterrotation along the longitudinal axis of the ultrasonic probe that creates a plurality of torsional nodes and a plurality of torsional anti-nodes along a portion of the longitudinal axis of the ultrasonic probe resulting in cavitation along a portion of the longitudinal axis of the ultrasonic probe comprising a radially asymmetric cross section that ablates the biological material.

### RELATED APPLICATIONS

None.

### FIELD OF THE INVENTION

The present invention relates to ultrasonic medical devices, and more particularly to an apparatus and method for an ultrasonic medical device operating in a torsional mode to ablate a biological material.

### BACKGROUND OF THE INVENTION

The presence of biological material in various parts of the human body can lead to complications ranging from artery disease, heart attack, stroke and in some cases death. The safe and effective destruction of the biological material that causes these complications is an important endeavor in the medical field. A variety of prior art instruments and methods destroy biological material in the human body.

Prior art medical instruments used to destroy biological material in the body suffer from several limitations. Prior art medical instruments are large, making it difficult for medical professionals to utilize them. Prior art medical instruments utilize high power levels that can adversely affect areas surrounding the treatment area and the patient. Procedures using prior art medical instruments are time consuming in comparison with other methods such as surgical excision.

Prior art medical instruments using ultrasonic energy have relied on longitudinal vibrations. By creating longitudinal vibrations, the prior art medical instruments remove the biological material through successive motion in a manner similar to a jackhammer. By vibrating longitudinally, the temperature of prior art medical instruments increases, thereby increasing the potential of necrosis of healthy tissue in the treatment area. The necrosis of otherwise healthy tissue in the region of the surgical procedure can complicate recovery and provide a potential site of post-operative infection. In many cases, the prior art instruments operating in a longitudinal mode remove small amounts of biological material and increase the overall time of the medical procedure.

For example, U.S. Pat. No. 4,961,424 to Kubota et al. discloses an ultrasonic treatment device operating in a longitudinal mode that is urged or brought into contact with an area to be treated. U.S. Pat. No. 4,870,953 to DonMicheal et al. discloses an intravascular ultrasonic catheter/probe and method for treating intravascular blockage that delivers ultrasonic energy via a longitudinal vibration. U.S. Pat. No. 5,391,144 to Sakurai et al. discloses an ultrasonic treatment apparatus that includes an instrument operating in a longitudinal mode that emulsifies tissue. Therefore, there remains a need in the art for a device operating in a torsional mode that can safely and effectively destroy a biological material in a time efficient manner.

Torsional mode vibration of objects is known in the art. However, the prior art does not describe the torsional mode vibration of a medical device. Further, the prior art requires additional objects to be attached to the prior art instruments, thereby preventing a minimally invasive solution of destroying biological material using torsional mode vibration. For example, U.S. Pat. No. 4,771,202 and U.S. Pat. No. 4,498,025 both to Takahashi disclose a tuning fork using the fundamental vibration of a flexural mode coupled with the fundamental mode of torsion. The fundamental frequency of the torsional mode is adjusted by placing masses near the side edges of the tine tips. U.S. Pat. No. 4,652,786 to Mishiro discloses a torsional vibration apparatus having a plurality of electrodes formed on the two surfaces of a circular member of electrostrictive material. Therefore, there remains a need in the art for a medical device that vibrates in a torsional mode to safely and effectively destroy biological material in the body in a time efficient manner.

The prior art does not provide a solution for destroying biological material in a safe, effective and time efficient manner. Prior art instruments utilizing longitudinal vibrations have difficulty distinguishing between healthy tissue and diseased tissue. Therefore, there remains a need in the art for an apparatus and a method for an ultrasonic medical device operating in a torsional mode to ablate biological material in a safe, effective and time efficient manner.

### SUMMARY OF THE INVENTION

The present invention is an apparatus and a method for an ultrasonic medical device operating in a torsional mode. The ultrasonic medical device comprises an ultrasonic probe having a proximal end, a distal end and a longitudinal axis therebetween; a transducer creating a torsional vibration of the ultrasonic probe; a coupling engaging the proximal end of the ultrasonic probe to a distal end of the transducer; and an ultrasonic energy source engaged to the transducer that produces an ultrasonic energy.

The present invention is a medical device comprising an elongated probe comprising a proximal end, a distal end and a longitudinal axis between the proximal end and the distal end where a portion of the longitudinal axis comprises a radially asymmetric cross section. The medical device includes a transducer that converts electrical energy into mechanical energy, creating a torsional vibration along the longitudinal axis of the elongated probe. A coupling engages the proximal end of the elongated probe to a distal end of the transducer. An ultrasonic energy source engaged to a proximal end of the transducer provides electrical energy to the transducer. The torsional vibration along the elongated probe produces a plurality of torsional nodes and a plurality of torsional anti-nodes along a portion of the longitudinal axis of the elongated probe.

The present invention is a method of treating a biological material in a body with an ultrasonic medical device comprising: providing an ultrasonic probe having a proximal end, a distal end and a longitudinal axis therebetween where a portion of the longitudinal axis comprises a radially asymmetric cross section; moving the ultrasonic probe to a treatment site of the biological material to place the ultrasonic probe in communication with the biological material; and actuating an ultrasonic energy source engaged to the ultrasonic probe to produce an ultrasonic energy that is converted into a torsional vibration of the ultrasonic probe.

The present invention is a method of removing a biological material in a body comprising providing an ultrasonic medical device comprising an ultrasonic probe having a proximal end, a distal end that terminates in a probe tip and a longitudinal axis between the proximal end and the distal end. The ultrasonic probe is moved in the body and placed in communication with the biological material. An ultrasonic energy source of the ultrasonic medical device is activated to produce an electrical signal that drives a transducer of the ultrasonic medical device to produce a torsional vibration of the ultrasonic probe. The torsional vibration of the ultrasonic probe produces a plurality of torsional nodes and a plurality of torsional anti-nodes along a portion of the longitudinal axis of the ultrasonic probe.

The present invention is an ultrasonic probe that supports a torsional vibration, comprising a proximal end, a distal end that terminates in a probe tip and a longitudinal axis between the proximal end and the distal end.

The present invention provides an apparatus and a method for an ultrasonic medical device operating in a torsional mode. An ultrasonic probe of the ultrasonic medical device undergoes a torsional vibration that produces a rotation and a counterrotation along the longitudinal axis of the ultrasonic probe. The torsional vibration of the ultrasonic probe is projected in a forward and a reverse direction about a plurality of torsional nodes along a portion of the longitudinal axis of the ultrasonic probe. The torsional vibration produces a rotation and a counterrotation along the longitudinal axis of the ultrasonic probe that creates a plurality of torsional nodes and a plurality of torsional anti-nodes along a portion of the longitudinal axis of the ultrasonic probe resulting in cavitation along an active area of the ultrasonic probe in a medium surrounding the ultrasonic probe that ablates the biological material. The present invention provides an ultrasonic medical device that is simple, user-friendly, time efficient, reliable and cost effective.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1 is a side plan view of an ultrasonic medical device of the present invention capable of operating in a torsional mode having two defined intervals with different cross sectional geometries.

FIG. 2 is a side plan view of an ultrasonic probe of the present invention having a uniform cross section from a proximal end of the ultrasonic probe to a distal end of the ultrasonic probe.

FIG. 3A is a perspective view of a portion of a longitudinal axis of an ultrasonic probe of the present invention with a distal end having a radially asymmetric cross section.

FIG. 3B is a cross section of a distal end of an ultrasonic probe of the present invention having a radially asymmetric cross section.

FIG. 3C is a cross section of a proximal end of an ultrasonic probe of the present invention having a radially symmetric cross section.

FIG. 4 is a fragmentary perspective view of the ultrasonic probe of the present invention undergoing torsional vibration.

FIG. 5A is a side plan view of the ultrasonic probe of the present invention undergoing torsional vibration.

FIG. 5B is a graph corresponding to the torsional vibration shown in FIG. 5A.

FIG. 6 is a fragmentary side plan view of the ultrasonic probe of the present invention having a plurality of torsional nodes and a plurality of torsional anti-nodes along a portion of the longitudinal axis of the ultrasonic probe.

FIG. 7 is a perspective view of an alternative embodiment of the present invention wherein an ultrasonic probe comprises a distal section having a spline shape.

FIG. 8 is a side plan view of an alternative embodiment of the present invention wherein an ultrasonic probe comprises a distal section having a spline shape.

FIG. 9 is a cross section taken along line A-A of FIG. 8 of a distal section of an ultrasonic probe of an alternative embodiment of the present invention.

FIG. 10 is a side plan view of the ultrasonic probe of the present invention within a sheath.

While the above-identified drawings set forth preferred embodiments of the present invention, other embodiments of the present invention are also contemplated, as noted in the discussion. This disclosure presents illustrative embodiments of the present invention by way of representation and not limitation. Numerous other modifications and embodiments can be devised by those skilled in the art which fall within the scope and spirit of the principles of the present invention.

### DETAILED DESCRIPTION

The present invention provides an apparatus and a method for using an ultrasonic medical device in a torsional mode to treat a biological material. The ultrasonic medical device comprises an ultrasonic probe, a transducer, a coupling engaging a proximal end of the ultrasonic probe to a distal end of the transducer and an ultrasonic energy source engaged to the transducer. The ultrasonic energy source produces an ultrasonic energy that is transmitted to the transducer, where the transducer creates a torsional vibration of the ultrasonic probe. The torsional vibration produces a rotation and a counterrotation along the longitudinal axis of the ultrasonic probe that creates a plurality of torsional nodes and a plurality of torsional anti-nodes along a portion of the longitudinal axis of the ultrasonic probe. A radially asymmetric cross section of a portion of a longitudinal axis of the ultrasonic probe supports the torsional vibration cavitation. The torsional vibration creates cavitation along the length with the radially asymmetric cross section (“an active area”) of the ultrasonic probe in a medium surrounding the ultrasonic probe that is displaced to ablate the biological material.

The following terms and definitions are used herein:

“Ablate” as used herein refers to removing, clearing, destroying or taking away a biological material. “Ablation” as used herein refers to a removal, clearance, destruction, or taking away of the biological material.

“Torsional Node” as used herein refers to a point along a longitudinal axis of an ultrasonic probe where the cross section does not rotate from a static position. The torsional node is a point of minimum rotational amplitude along the longitudinal axis of the ultrasonic probe.

“Torsional Anti-node” as used herein refers to a point along a longitudinal axis of an ultrasonic probe where an angular displacement of a cross section from a static position is locally a maximum. The torsional anti-node is a point of a maximum rotational amplitude of the ultrasonic probe.

“Probe” as used herein refers to a device capable of propagating an energy emitted by the ultrasonic energy source along a longitudinal axis of the ultrasonic probe, resolving the energy into an effective cavitational energy at a specific resonance (defined by a plurality of nodes and a plurality of anti-nodes along an “active area” of the probe) and is capable of an acoustic impedance transformation of an ultrasound energy to a mechanical energy.

“Biological material” as used herein refers to a collection of a matter including, but not limited to, a group of similar cells, intravascular blood clots or thrombus, fibrin, calcified plaque, calcium deposits, occlusional deposits, atherosclerotic plaque, fatty deposits, adipose tissues, atherosclerotic cholesterol buildup, fibrous material buildup, arterial stenoses, minerals, high water content tissues, platelets, cellular debris, wastes and other occlusive materials.

“Torsional vibration” as used herein refers to torsional movement wherein portions of an object move alternately in opposite directions from a position of equilibrium. Torsional vibration also refers to torsional motion, torsional oscillation and propagation of torsional waves.

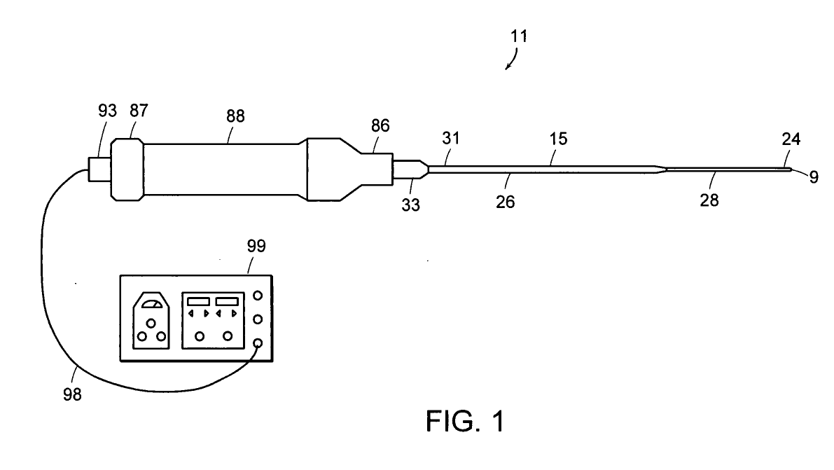

An ultrasonic medical device capable of operating in a torsional mode is illustrated generally at 11 in FIG. 1. The ultrasonic medical device 11 includes an ultrasonic probe 15 which is coupled to an ultrasonic energy source or generator 99 for the production of an ultrasonic energy. A handle 88, comprising a proximal end 87 and a distal end 86, surrounds a transducer within the handle 88. The transducer, having a proximal end engaging the ultrasonic energy source 99 and a distal end coupled to a proximal end 31 of the ultrasonic probe 15, transmits the ultrasonic energy to the ultrasonic probe 15. A connector 93 and a connecting wire 98 engage the ultrasonic energy source 99 to the transducer. The ultrasonic probe 15 includes the proximal end 31, a distal end 24 that ends in a probe tip 9 and a longitudinal axis between the proximal end 31 and the distal end 24. In a preferred embodiment of the present invention shown in FIG. 1, a cross section of the ultrasonic probe transitions from an approximately circular cross section at a first defined interval 26 to a radially asymmetric cross section at a second defined interval 28 along the longitudinal axis of the ultrasonic probe 15 over a transition 82. A coupling 33 that engages the proximal end 31 of the ultrasonic probe 15 to the transducer within the handle 88 is illustrated generally in FIG. 1. In a preferred embodiment of the present invention, the coupling is a quick attachment-detachment system. An ultrasonic medical device with a quick attachment-detachment system is described in the Assignee's co-pending patent applications U.S. Ser. No. 09/975,725; U.S. Ser. No. 10/268,487 and U.S. Ser. No. 10/268,843, and the entirety of all these applications are hereby incorporated herein by reference.

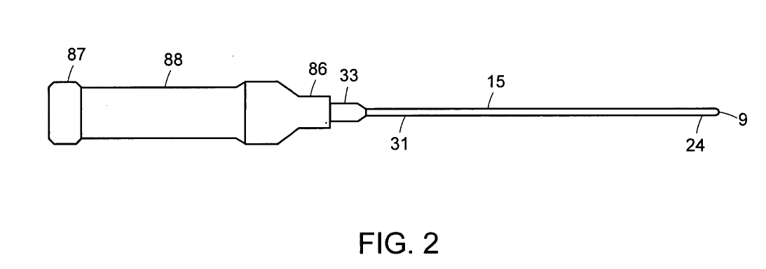

FIG. 2 shows an alternative embodiment of the ultrasonic probe 15 of the present invention. In the embodiment of the present invention shown in FIG. 2, the cross section of the ultrasonic probe 15 is approximately uniform from the proximal end 31 of the ultrasonic probe 15 to the distal end 24 of the ultrasonic probe 15.

In a preferred embodiment of the present invention, the ultrasonic probe 15 is a wire. In an embodiment of the present invention, the ultrasonic probe 15 is elongated. In an embodiment of the present invention, the cross section of the ultrasonic probe 15 changes at greater than two defined intervals. In an embodiment of the present invention, the transitions 82 of the ultrasonic probe 15 are tapered to gradually change the cross section from the proximal end 31 to the distal end 24 along the longitudinal axis of the ultrasonic probe 15. In another embodiment of the present invention, the transitions 82 of the ultrasonic probe 15 are stepwise to change the cross section from the proximal end 31 to the distal end 24 along the longitudinal axis of the ultrasonic probe 15. Those skilled in the art will recognize that there can be any number of defined intervals and transitions, and that the transitions can be of any shape known in the art and be within the spirit and scope of the present invention.

In an embodiment of the present invention, the gradual change of the cross section from the proximal end 31 to the distal end 24 occurs over the at least one transition 82, with each transition 82 having an approximately equal length. In another embodiment of the present invention, the gradual change of the cross section from the proximal end 31 to the distal end 24 occurs over a plurality of transitions 82 with each transition 82 having a varying length. The transition 82 refers to a section where the cross section varies from a first cross section to a second cross section.

The probe tip 9 can be any shape including, but not limited to, bent, a ball or larger shapes. In one embodiment of the present invention, the ultrasonic energy source 99 is a physical part of the ultrasonic medical device 11. In another embodiment of the present invention, the ultrasonic energy source 99 is not an integral part of the ultrasonic medical device 11. The ultrasonic probe 15 is used to treat a biological material and may be disposed of after use. In a preferred embodiment of the present invention, the ultrasonic probe 15 is for a single use and on a single patient. In a preferred embodiment of the present invention, the ultrasonic probe 15 is disposable. In another embodiment of the present invention, the ultrasonic probe 15 can be used multiple times.

The ultrasonic probe 15 has a stiffness that gives the ultrasonic probe 15 a flexibility allowing the ultrasonic probe 15 to be deflected and articulated when the ultrasonic medical device 11 is in motion. The ultrasonic probe 15 can be bent, flexed and deflected to reach the biological material at locations in the vasculature of the body that are difficult to reach. The ultrasonic probe 15 is designed, constructed and comprised of a material to not dampen the torsional vibration, and thereby supports a torsional vibration when flexed. The flexibility of the ultrasonic probe 15 is advantageous when the ultrasonic probe 15 is inserted into a tortuous path of a vasculature of a mammal.

In a preferred embodiment of the present invention, the ultrasonic probe 15 comprises titanium or a titanium alloy. In a preferred embodiment of the present invention, the ultrasonic probe 15 comprises titanium alloy Ti-6Al-4V. The elements comprising Ti-6Al-4V and the representative elemental weight percentages of Ti-6Al-4V are titanium (about 90%), aluminum (about 6%), vanadium (about 4%), iron (maximum about 0.25%) and oxygen (maximum about 0.2%). Titanium is a strong, flexible, low density, low radiopacity and easily fabricated metal that is used as a structural material. Titanium and its alloys have excellent corrosion resistance in many environments and have good elevated temperature properties. In another embodiment of the present invention, the ultrasonic probe 15 comprises stainless steel. In another embodiment of the present invention, the ultrasonic probe 15 comprises an alloy of stainless steel. In another embodiment of the present invention, the ultrasonic probe 15 comprises aluminum. In another embodiment of the present invention, the ultrasonic probe 15 comprises an alloy of aluminum. In another embodiment of the present invention, the ultrasonic probe 15 comprises a combination of titanium and stainless steel. Those skilled in the art will recognize that the ultrasonic probe can be comprised of many other materials known in the art and be within the spirit and scope of the present invention.

The physical properties (i.e. length, cross sectional shape, dimensions etc.) and material properties (i.e., yield strength, modulus, etc.) of the ultrasonic probe 15 are selected for operation of the ultrasonic probe 15 in the torsional mode. The length of the ultrasonic probe 15 of the present invention is chosen so as to be resonant in a torsional mode. In an embodiment of the present invention, the ultrasonic probe 15 is between about 30 centimeters and about 300 centimeters in length. For the ultrasonic probe 15 to operate in a pure torsional mode, the length of the ultrasonic probe 15 should be an integer multiple of one-half wavelength of the drive transducer's torsional resonance. The section below entitled “Theory of Operation” provides details and equations for determining the length for the ultrasonic probe operating in a torsional mode. For example, for an ultrasonic probe comprised of titanium operating at a frequency of 20 kHz, the length of the ultrasonic probe should be an integer multiple of one-half wavelength (approximately 7.58 centimeters (about 3 inches)). Those skilled in the art will recognize an ultrasonic probe can have a length shorter than about 30 centimeters, a length longer than about 300 centimeters and a length between about 30 centimeters and about 300 centimeters and be within the spirit and scope of the present invention.

The handle 88 surrounds the transducer located between the proximal end 31 of the ultrasonic probe 15 and the connector 93. In a preferred embodiment of the present invention, the transducer includes, but is not limited to, a horn, an electrode, an insulator, a backnut, a washer, a piezo microphone, and a piezo drive. The transducer converts electrical energy provided by the ultrasonic energy source 99 to mechanical energy and sets the operating frequency of the ultrasonic medical device 11. By an appropriately oriented and driven cylindrical array of piezoelectric crystals of the transducer, the horn creates a torsional wave along at least a portion of the longitudinal axis of the ultrasonic probe 15, causing the ultrasonic probe 15 to vibrate in a torsional mode. The transducer crystals are vibrated in a direction approximately tangential to the cylindrical surface of the ultrasonic probe 15. U.S. Pat. No. 2,838,695 to Thurston describes how an appropriately oriented and driven cylindrical array of transducer crystals creates torsional waves, and the entirety of this patent is hereby incorporated herein by reference. The transducer is capable of engaging the ultrasonic probe 15 at the proximal end 31 with sufficient restraint to form an acoustical mass that can propagate the ultrasonic energy provided by the ultrasonic energy source 99.

The ultrasonic energy source 99 produces the electrical energy that is used to produce a torsional vibration along a portion of the longitudinal axis of the ultrasonic probe 15. The ultrasonic energy source 99 provides the electrical power to the transducer at the resonant frequency of the transducer. In a preferred embodiment of the present invention, the ultrasonic energy source 99 finds the resonant frequency of the transducer through a Phase Lock Loop (PLL) circuit. The ultrasonic probe 15 can support the torsional vibration along the longitudinal axis of the ultrasonic probe 15.

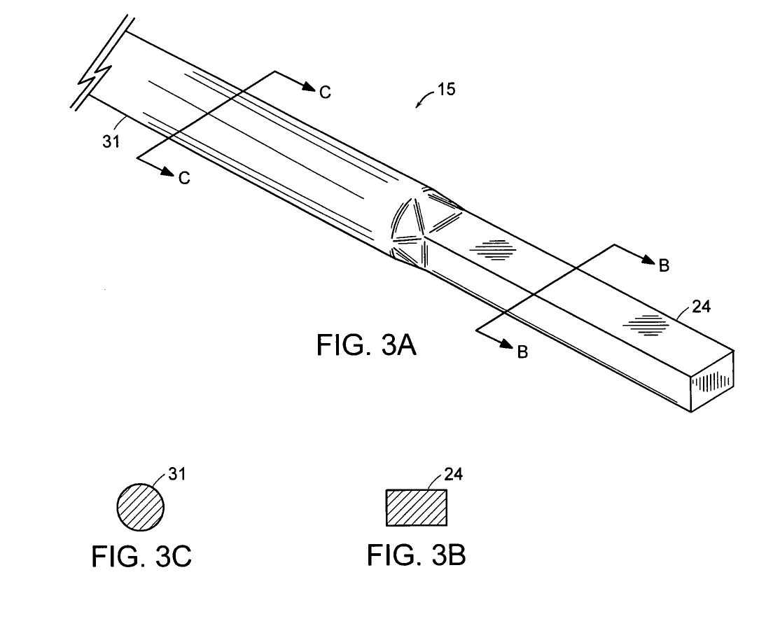

FIG. 3A shows a preferred embodiment of a portion of the longitudinal axis of the ultrasonic probe 15 of the present invention. In the preferred embodiment of the present invention shown in FIG. 3A, the proximal end 31 of the ultrasonic probe 15 comprises an approximately circular cross section and the distal end 24 comprises a radially asymmetric cross section. FIG. 3B is a cross section of the distal end 24 of the ultrasonic probe 15, as taken from line B-B of FIG. 3A. FIG. 3C is a cross section of the proximal end 31 of the ultrasonic probe 15, as taken from line C-C of FIG. 3A.

In a preferred embodiment of the present invention shown in FIG. 3B, a cross section of the distal end 24 of the ultrasonic probe 15 is radially asymmetric. In a most preferred embodiment of the present invention, the radially asymmetric cross section at the distal end 24 is approximately rectangular shaped. In another embodiment of the present invention, the radially asymmetric cross section at the distal end 24 of the ultrasonic probe includes, but is not limited to, square shaped, oval shaped, trapezoidal, star shaped, circular with a flat spot, elliptical and triangular. Those skilled in the art will recognize that other radially asymmetric cross sectional geometries known in the art would be within the spirit and scope of the present invention.

As will be described in further detail below, the radially asymmetric cross section of the ultrasonic probe 15 vibrating in the torsional mode efficiently transfers an acoustic energy to the medium surrounding the ultrasonic probe 15. By transferring the acoustic energy to the medium surrounding the ultrasonic probe 15, cavitation is created over the length of the radially asymmetric cross section of the ultrasonic probe 15. The length of the radially asymmetric cross section creates an active area of the ultrasonic probe 15 over which ablation of a biological material occurs.

In a preferred embodiment of the present invention shown in FIG. 3C, the cross section of the proximal end of the ultrasonic probe 15 is approximately circular. In a preferred embodiment of the present invention, the diameter of the proximal end 31 of the ultrasonic probe 15 is about 0.012 inches. In another embodiment of the present invention, the diameter of the proximal end 31 of the ultrasonic probe 15 is about 0.025 inches. In other embodiments of the present invention, the diameter of the proximal end 31 of the ultrasonic probe 15 varies between about 0.003 inches and about 0.025 inches. Those skilled in the art will recognize the ultrasonic probe 15 can have a diameter at the proximal end 31 smaller than about 0.003 inches, larger than about 0.025 inches, and between about 0.003 inches and about 0.025 inches and be within the spirit and scope of the present invention.

The torsional mode of vibration of the ultrasonic probe 15 according to the present invention differs from an axial (or longitudinal) mode of vibration disclosed in the prior art. Rather than vibrating in an axial direction, the ultrasonic probe 15 of the present invention vibrates torsionally. The torsional vibration produces a plurality of torsional nodes 50 and a plurality of torsional anti-nodes 52 along a portion of the longitudinal axis of the ultrasonic probe 15. The biological material is removed in a region having a radius of up to about 6 mm along the portion of the longitudinal axis of the ultrasonic probe 15 having the radially asymmetric cross section.

The ultrasonic probe 15 is moved to a treatment site of the biological material and the ultrasonic probe 15 is placed in communication with the biological material. The ultrasonic probe 15 may be swept, twisted or rotated along the treatment site of the biological material. Those skilled in the art will recognize the ultrasonic probe can be placed in communication with the biological material in many other ways known in the art and be within the spirit and scope of the present invention.

The ultrasonic energy source 99 is activated to produce the ultrasonic energy that is subsequently converted into the torsional vibration of the ultrasonic probe 15. The operating frequency of the ultrasonic medical device 11 is set by the transducer and the ultrasonic energy source 99 finds the resonant frequency of the transducer through the Phase Lock Loop. The ultrasonic energy source 99 provides a low power electric signal of between about 2 watts to about 15 watts to the transducer that is located within the handle 88. The transducer converts electrical energy provided by the ultrasonic energy source to mechanical energy. The piezoelectric ceramic crystals of the transducer are vibrated in the direction approximately tangential to the surface and approximately perpendicular to the longitudinal axis of the ultrasonic probe 15, creating the torsional wave that is transmitted along the longitudinal axis of the ultrasonic probe 15.

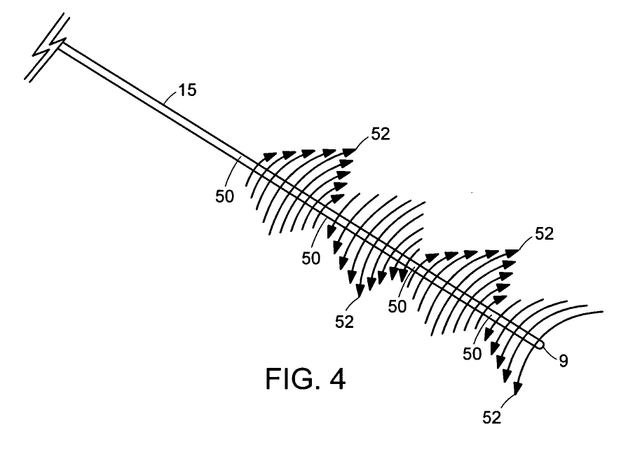

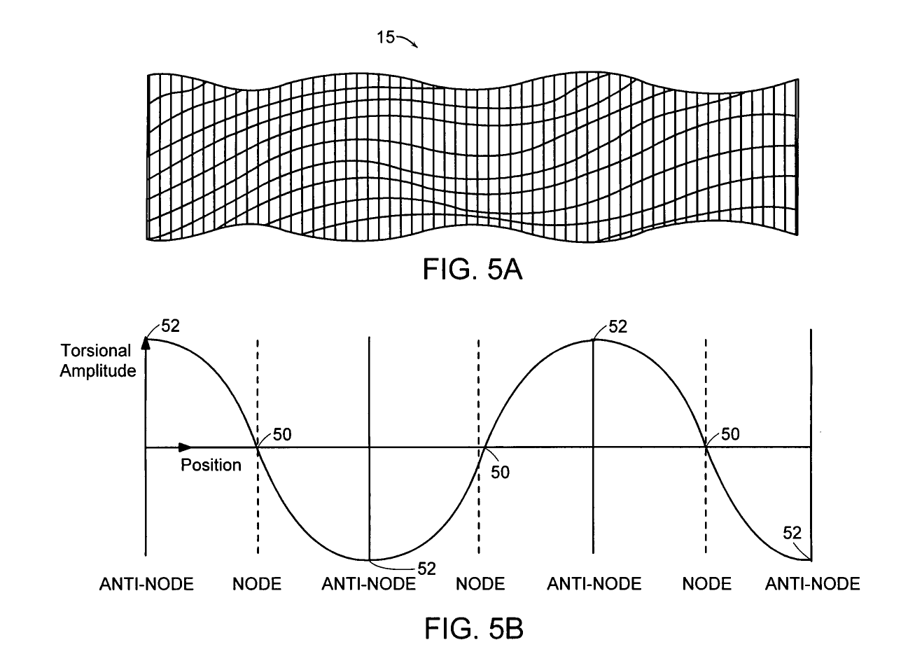

FIG. 4 shows a fragmentary perspective view of the ultrasonic probe 15 of the present invention undergoing the torsional vibration. FIG. 5A shows a side plan view of the ultrasonic probe of the present invention undergoing the torsional vibration while FIG. 5B shows a graph corresponding to the torsional vibration shown in FIG. 5A. FIG. 5B shows the location of torsional nodes 50 and torsional anti-nodes 52 when the ultrasonic probe 15 undergoes the torsional motion. The torsional vibration of the ultrasonic probe 15 in FIG. 4 and FIG. 5A is shown as movement of the ultrasonic probe in alternating clockwise and counterclockwise directions along the longitudinal axis of the ultrasonic probe 15. As shown in FIG. 5A and FIG. 5B, the torsional vibration is propagated in a forward direction and a reverse direction about the torsional node 50. At each torsional node 50, the direction of the rotation reverses and the amplitude increases until reaching the torsional anti-node 52 and subsequently decreases toward the next torsional node 50.

FIG. 5A shows the alternating clockwise motion and counterclockwise motion about the torsional node 50 and shows an expansion and a compression of the ultrasonic probe in the torsional mode. FIG. 5A shows the expansion of the ultrasonic probe 15 as the clockwise and counterclockwise motion of the ultrasonic probe 15 extends away from the torsional node 50. As the alternating clockwise and counterclockwise motion returns back to the torsional node 50, the ultrasonic probe 15 is compressed. The ultrasonic probe 15 will expand and compress about the plurality of torsional nodes 50 along a portion of the longitudinal axis of the ultrasonic probe 15.

The torsional wave is transmitted along the longitudinal axis of the ultrasonic probe 15 and the interaction of the surface of the ultrasonic probe 15 with the medium surrounding the ultrasonic probe 15 creates an acoustic wave in the surrounding medium. As the torsional wave is transmitted along the longitudinal axis of the ultrasonic probe 15, the ultrasonic probe 15 vibrates torsionally. The efficiency with which the energy of the torsional vibration of the ultrasonic probe 15 is transmitted to the medium surrounding the ultrasonic probe 15 is dependent on the shape of the cross section of the ultrasonic probe 15.

As discussed above, in a preferred embodiment of the present invention the cross section of the distal end 24 of the ultrasonic probe 15 is radially asymmetric. In another embodiment of the present invention, approximately the entire length of the ultrasonic probe 15 comprises a radially asymmetric cross section. In a most preferred embodiment of the present invention, the cross section of the distal end 24 of the ultrasonic probe 15 is approximately rectangular shaped. For the radially asymmetric cross section of the ultrasonic probe 15, the torsional wave transmitted along the longitudinal axis of the ultrasonic probe 15 produces a component of motion approximately perpendicular to the interface between the ultrasonic probe 15 and the medium surrounding the ultrasonic probe 15. By producing a component of motion approximately perpendicular to this interface, the acoustic energy is transferred to the medium surrounding the ultrasonic probe 15.

Torsional motion of the ultrasonic probe produces cavitation in the medium surrounding the ultrasonic probe to ablate the biological material along the portion of the longitudinal axis of the ultrasonic probe having the radially asymmetric cross section. Cavitation is a process in which small voids are formed in a surrounding medium through the rapid motion of the ultrasonic probe 15 and the voids are subsequently forced to compress. The compression of the voids creates a wave of acoustic energy which acts to dissolve the matrix binding the biological material, while having no damaging effects on healthy tissue.

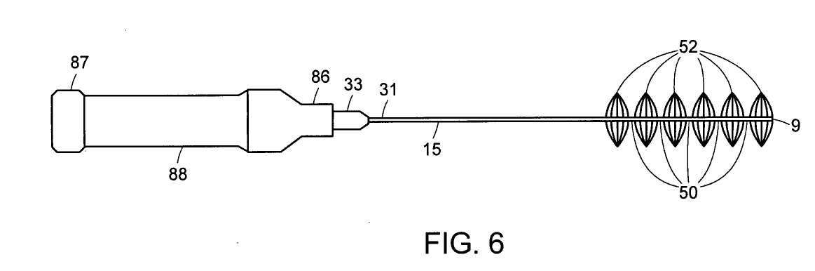

FIG. 6 shows a fragmentary side plan view of the ultrasonic probe 15 of the present invention showing the plurality of torsional nodes 50 and the plurality of torsional anti-nodes 52 along a portion of the longitudinal axis of the ultrasonic probe 15. The torsional nodes 50 are areas of minimum rotational amplitude and minimum vibration. A plurality of torsional anti-nodes 52, areas of maximum rotational amplitude and maximum vibration, also occur at repeating intervals along a portion of the longitudinal axis of the ultrasonic probe 15. The number of torsional nodes 50 and the number of torsional anti-nodes 52 occurring along the longitudinal axis of the ultrasonic probe 15 is modulated by changing the frequency of energy supplied by the ultrasonic energy source 99. The exact frequency, however, is not critical and the ultrasonic energy source 99 run at, for example, about 20 kHz is sufficient to create an effective number of biological material destroying torsional anti-nodes 52 along the active area of the ultrasonic probe 15. The low frequency requirement of the present invention is a further advantage in that the low frequency requirement leads to less damage to healthy tissue. Those skilled in the art will recognize that changing the dimensions of the ultrasonic probe 15, including diameter, length and distance to the ultrasonic energy source 99, will affect the number and spacing of the torsional nodes 50 and the torsional anti-nodes 52 along a portion of the longitudinal axis of the ultrasonic probe 15.

The present invention allows the use of ultrasonic energy to be applied to the biological material selectively, because the ultrasonic probe 15 conducts energy across a frequency range from about 10 kHz through about 100 kHz. The amount of ultrasonic energy to be applied to a particular treatment site is a function of the amplitude and frequency of vibration of the ultrasonic probe 15. In general, the amplitude or throw rate of energy is in the range of about 25 microns to about 250 microns, and the frequency in the range of about 10 kHz to about 100 kHz. In a preferred embodiment of the present invention, the frequency of ultrasonic energy is from about 20 kHz to about 35 kHz.

With the ultrasonic probe 15 in communication with the biological material and the ultrasonic energy source 99 activated, the ultrasonic probe 15 undergoes the torsional vibration. The torsional vibration of the ultrasonic probe 15 causes a rotation and counterrotation along the longitudinal axis of the ultrasonic probe 15. The torsional vibration of the ultrasonic probe 15 is propagated in a forward direction and a reverse direction about the plurality of torsional nodes 50 along a portion of the longitudinal axis, creating cavitation along the portion of the longitudinal axis of the ultrasonic probe 15 having the radially asymmetric cross section that ablates the biological material. In an alternative embodiment of the present invention, the length over which cavitation occurs can be increased by increasing the power of the ultrasonic medical device 11.

The torsional mode of vibration is a torsional oscillation whereby equally spaced points along the longitudinal axis of the ultrasonic probe 15 including the probe tip 9 vibrate back and forth in a short arc about the longitudinal axis of the ultrasonic probe 15. A section proximal to each of the plurality of torsional nodes 50 and a section distal to each of the plurality of torsional nodes 50 are vibrated out of phase, with the proximal section vibrated in a clockwise direction and the distal section vibrated in a counterclockwise direction, or vice versa. The proximal section to each of the plurality of torsional nodes 50 and the distal section to each of the plurality of torsional nodes 50 are subsequently vibrated in opposite directions to create a torsional motion of the ultrasonic probe 15. The torsional mode of vibration results in an ultrasonic energy transfer to the biological material with minimal loss of ultrasonic energy that could limit the effectiveness of the ultrasonic medical device 11. The torsional mode of vibration results in biological material being ablated in a time efficient manner along the portion of the longitudinal axis of the ultrasonic probe having the radially asymmetric cross section. By operating in a torsional mode of vibration, the ultrasonic probe 15 rotates back and forth in clockwise and counterclockwise directions, creating a plurality of torsional nodes 50 and a plurality of torsional anti-nodes 52 along a portion of the longitudinal axis of the ultrasonic probe 15 resulting in cavitation along the portion of the longitudinal axis of the ultrasonic probe having the radially asymmetric cross section in a medium surrounding the ultrasonic probe 15 to ablate the biological material.

As the ultrasonic probe 15 vibrates torsionally as shown in FIG. 4 and FIG. 5A, the torsional vibration creates cavitation along the portion of the longitudinal axis of the ultrasonic probe 15 having the radially asymmetric cross section in a medium surrounding the ultrasonic probe 15. The biological material is resolved into a particulate having a size on the order of red blood cells (approximately 5 microns in diameter). The size of the particulate is such that the particulate is easily discharged from the body through conventional methods or simply dissolves into the blood stream. A conventional method of discharging the particulate from the body includes transferring the particulate through the blood stream to the kidney where the particulate is excreted as bodily waste.

As discussed above, other radially asymmetric cross sections at the distal end 24 of the ultrasonic probe 15 can be used so that the torsional motion of the ultrasonic probe 15 produces a component of motion approximately perpendicular to the interface between the ultrasonic probe 15 and the medium surrounding the ultrasonic probe 15. The radially asymmetric cross sectional geometry enables the acoustic energy to be most efficiently transferred to the medium surrounding the ultrasonic probe 15. The biological material destroying effect of the ultrasonic probe 15 operating in the torsional mode of vibration is enhanced by having the cross sectional shape to be radially asymmetric. By increasing the length over which a radially asymmetric cross section spans, the active area over which cavitation and the biological material ablation occurs increases. The radial asymmetric cross sectional shape causes sufficient displacement of the medium surrounding the area comprising the radial asymmetric shape to create cavitation. Various radially asymmetric cross sectional shapes including, but not limited to square, trapezoidal, elliptical, star shaped, rectangular, oval, triangular, circular with a flat spot and similar cross sections can be used to produce cavitation along the area comprising the varied radial asymmetric cross sectional shape.

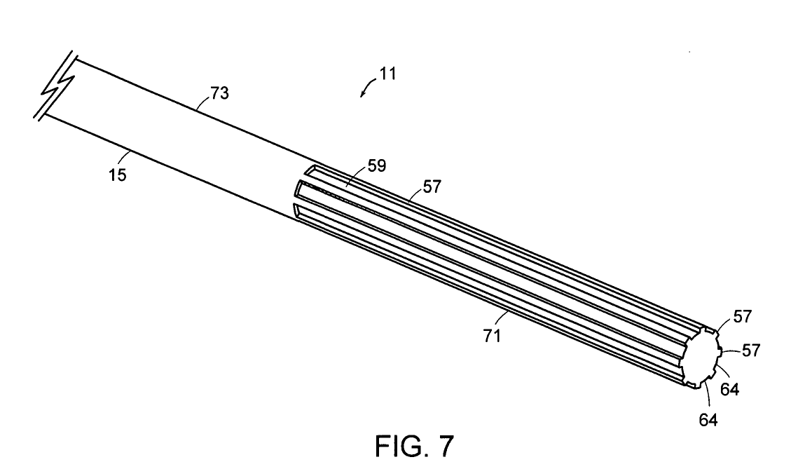

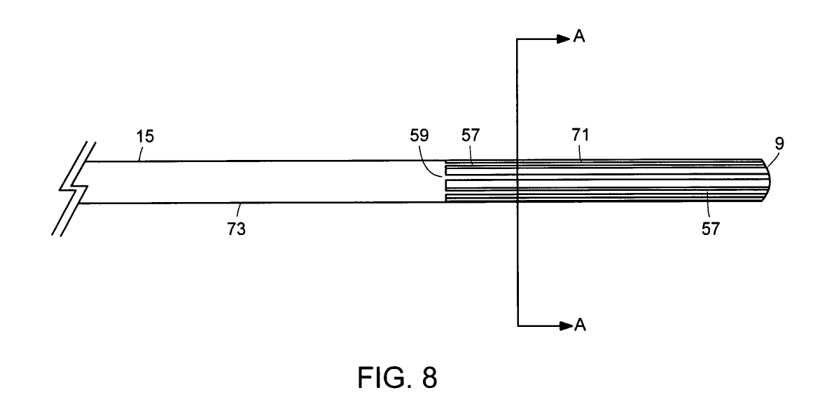

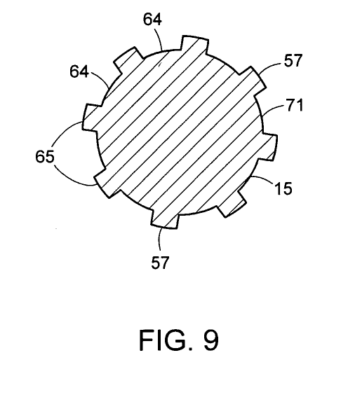

FIG. 7 shows a perspective view of an alternative embodiment of the present invention wherein the ultrasonic probe 15 comprises a radially asymmetric distal section 71 having a spline shape. A proximal section 73 of the ultrasonic probe 15 comprises an approximately circular cross section. FIG. 8 shows a side plan view of the ultrasonic probe 15 with the radially asymmetric distal section 71 having a plurality of projections. FIG. 9 shows a cross section taken along line A-A of FIG. 8 in the distal section 71 of the ultrasonic probe 15.

The radially asymmetric spline shaped cross section of the ultrasonic probe 15 shown in FIGS. 7-9 produces a component of motion of the ultrasonic probe 15 approximately perpendicular to the interface between the ultrasonic probe 15 and the medium surrounding the ultrasonic probe 15. As shown in FIGS. 7-9, the spline shape comprises a plurality of projections 57 extending from an outer surface 59 of the distal section 71 of the ultrasonic probe 15. The plurality of projections 57 may extend over varying lengths of the ultrasonic probe 15 creating varying active lengths over which cavitation occurs. The plurality of projections cause sufficient displacement of the surrounding medium to create cavitation. As best shown in FIG. 8, a plateau 64 is located between each of the plurality of projections 57. A diameter of the ultrasonic probe 15 at the plateau 64 may be equal to, less than or greater than the diameter of the ultrasonic probe 15 at a proximal section 73 of the ultrasonic probe 15. An end 65 of each of the plurality of projections 57 is contoured to minimize trauma to the biological material. The interaction between the plurality of projections 57 and the surrounding medium allows cavitation along the surface where the plurality of projections are present.

The spline shape enables energy to be more efficiently transferred to the medium surrounding the ultrasonic probe 15 and, as a result, increases the length of the active area over which cavitation and the biological material ablation occurs. The addition of the plurality of projections 57 maximizes the surface area of the ultrasonic probe 15 interacting with the surrounding medium and optimizes the viscous drag and therefore the stresses acting on the medium. The plurality of projections 57 cause sufficient displacement of the medium surrounding the plurality of projections 57 to cause cavitation along the plurality of projections 57. Those skilled in the art will recognize modifications of the cross sectional area resulting in a large surface area interacting with the medium would increase the length of the active area over which cavitation occurs and be within the spirit and scope of the present invention.

In an alternative embodiment of the present invention, the ultrasonic probe comprises an approximately circular cross section from the proximal end 31 to the distal end 24 of the ultrasonic probe 15. In an embodiment of the present invention, the diameter of the approximately circular cross section of the ultrasonic probe 15 decreases from the proximal end 31 to the distal end 24 of the ultrasonic probe 15. A torsionally vibrating ultrasonic probe with an approximately circular cross section has a surface moving approximately parallel to the interface between the ultrasonic probe 15 and the medium surrounding the ultrasonic probe 15. In this case, a shear wave is created in the medium surrounding the ultrasonic probe 15.

The propagation of shear waves in a fluid is significantly less than waves traveling approximately perpendicular to the surface of the ultrasonic probe. As a result, an approximately circular cross section is less effective for transferring the torsional vibration to the surrounding fluid. The area over which cavitation and ablation of the biological material occurs is at the probe tip 9 for the ultrasonic probe having an approximately circular cross section from the proximal end 31 to the distal end 24.

In the embodiment of the present invention where the cross section of the ultrasonic probe 15 is approximately circular, the diameter of the distal end 24 of the ultrasonic probe 15 is about 0.004 inches. In another embodiment of the present invention, the diameter of the distal end 24 of the ultrasonic probe 15 is about 0.015 inches. In other embodiments of the present invention, the diameter of the distal end 24 of the ultrasonic probe 15 varies between about 0.003 inches and about 0.025 inches. Those skilled in the art will recognize an ultrasonic probe 15 can have a diameter at the distal end 24 smaller than about 0.003 inches, larger than about 0.025 inches, and between about 0.003 inches and about 0.025 inches and be within the spirit and scope of the present invention.

The ultrasonic probe 15 can be fabricated with the varying cross sectional geometry by machining. In another embodiment of the present invention, a section comprising a varying cross section is engaged to an adjacent section of a different cross section along the longitudinal axis of the ultrasonic probe 15. The section comprising the varying cross section is engaged to the adjacent section of a different cross section by processes including, but not limited to, butt-welding, brazing, shrink fitting, lap welding, threaded fitting, twisting the materials, fastening or other mechanical or metallurgical connections. Those skilled in the art will recognize the ultrasonic probe can comprise a plurality of sections of varying cross sections and be fabricated in many ways known in the art and be within the spirit and scope of the present invention.

The section below entitled “Theory of Operation” discusses some differences between the longitudinal mode of operation used in the prior art and the torsional mode of operation used in the present invention. The ultrasonic medical device 11 of the present invention allows for shorter medical procedures. By reducing the time of the medical procedure, a patient is not subjected to additional health risks associated with longer medical procedures.

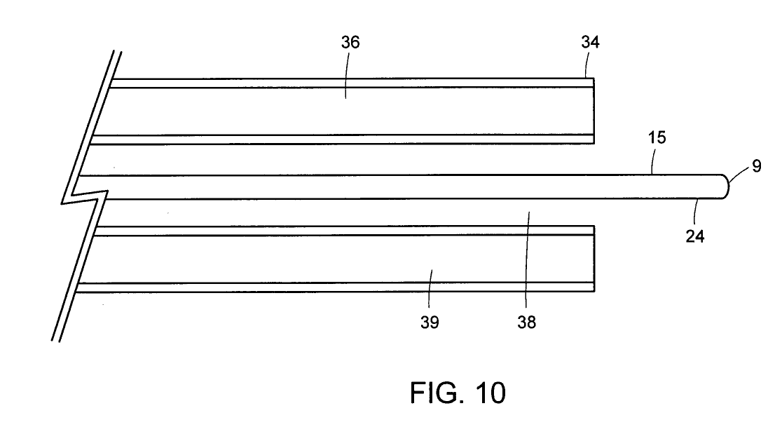

FIG. 10 shows the ultrasonic probe 15 of the present invention extending from a distal end 34 of a sheath 36. As shown in FIG. 10, the ultrasonic probe 15 is placed within the sheath 36, which can provide an at least one irrigation channel 38 and an at least one aspiration channel 39. In an embodiment of the present invention, irrigation is provided between the ultrasonic probe 15 and the sheath 36. The ultrasonic probe 15 may be moved in an axial direction within the sheath 36 to move the distal end 24 of the ultrasonic probe 15 axially inwardly and outwardly relative to the distal end 34 of the sheath 36. By extending or retracting the ultrasonic probe 15 relative to the sheath 36, the amount of the ultrasonic probe 15 exposed is modified, thereby modifying the biological material destroying effects of the ultrasonic probe 15.

In an embodiment of the present invention, the sheath 36 is comprised of polytetrafluoroethylene (PTFE). In another embodiment of the present invention, the sheath 36 is comprised of teflon tubing or similar fluoropolymer tubing. The sheath absorbs the ultrasonic energy emanating from the portions of the ultrasonic probe 15 located within the sheath 36, thereby allowing control over the amount of biological material affected by the ultrasonic probe 15. The sheath 36 is preferably comprised of a material which is resistant to heat from the ultrasonic energy, even though the irrigation fluid can act as a coolant for the sheath 36.

The present invention provides a method of treating a biological material in the body with the ultrasonic medical device 11. The ultrasonic probe 15 of the ultrasonic medical device 11 is moved to the treatment site of the biological material and placed in communication with the biological material. The ultrasonic energy source 99 of the ultrasonic medical device 11 engaged to the ultrasonic probe 15 is activated to produce the torsional vibration of the ultrasonic probe 15. The transducer engaging the ultrasonic energy source 99 at the proximal end of the transducer and the ultrasonic probe 15 at the distal end of the transducer creates the torsional vibration along the longitudinal axis of the ultrasonic probe 15. The torsional vibration of the ultrasonic probe 15 produces the plurality of torsional nodes 50 and the plurality of torsional anti-nodes 52 along a portion of the longitudinal axis of the ultrasonic probe 15. A portion of the longitudinal axis comprising the radially asymmetric cross section transfers the acoustic energy to the medium surrounding the ultrasonic probe 15, generating cavitation over the length with the radially asymmetric cross section, causing ablation of the biological material.

The present invention also provides a method of removing a biological material in the body. The ultrasonic probe 15 of the ultrasonic medical device 11 is moved in the body and placed in communication with the biological material. The ultrasonic energy source 99 of the ultrasonic medical device 11 produces an electric signal that drives the transducer of the ultrasonic medical device 11 to produce a torsional vibration of the ultrasonic probe 15. The torsional vibration of the ultrasonic probe 15 produces the plurality of torsional nodes 50 and the plurality of torsional anti-nodes 52 along a portion of the longitudinal axis of the ultrasonic probe 15. The torsional vibration of the ultrasonic probe 15 produces a rotation and a counterrotation along the longitudinal axis of the ultrasonic probe 15. The torsional vibration of the ultrasonic probe 15 is projected in a forward direction and a reverse direction about the plurality of torsional nodes 50 of the ultrasonic probe 15. The torsional vibration creates cavitation along a portion of the longitudinal axis of the ultrasonic probe 15 having the radially asymmetric cross section, resolving the biological material to a particulate having a size that can be easily discharged from the body through conventional means.

### Theory of Operation

The torsional mode of vibration of the present invention differs from longitudinal mode of vibration of the prior art. In the longitudinal vibration of the prior art, the frequencies of the individual modes depend on the modulus of elasticity E and the density ρ:

$${c}_{l} = \sqrt{\frac{E}{ρ}}$$

For the torsional waves, the expression is the same except the shear modulus, G, is used instead of the modulus of elasticity, E. The shear modulus, G, and the modulus of elasticity, E, are linked through Poisson's ratio υ:

$$G = \frac{E}{2 ( 1 + υ )}$$

and the corresponding torsional speed of propagation is:

$${c}_{t} = \sqrt{\frac{{GK}_{T}}{ρ I}}$$

where K_{T} is the torsional stiffness factor of the cross section and I is the moment of inertia of the cross section. For a circular cross section, the ratio K_{T}/I=1, while for radially asymmetric cross sections the ratio K_{T}/I<1. Therefore, the speed of propagation will be slower for the torsional wave by a factor of:

$$\frac{{c}_{t}}{{c}_{l}} = \sqrt{\frac{{K}_{T}}{2 ( 1 + υ ) I}}$$

For a symmetric cross section K_{T}/I=1, and for a radially asymmetric cross section K_{T}/I<1. For common metals, Poisson's ratio ν is on the order of 0.3, therefore the speed of propagation for a torsional wave on a body of circular cross section will be approximately 62% or less of that for the longitudinal wave. A decrease in the speed of propagation implies a proportional decrease in the wavelength for a given frequency. Decreasing the wavelength greatly improves the device's ability to deliver energy through the tortuous paths and the tight bends of the vasculature.

The operating frequencies of the longitudinal and torsional modes are dependent on the properties of the ultrasonic probe. Selection of material properties depends primarily on the choice of operating frequency and the desired amplitude of vibration. As discussed previously, with the ultrasonic probe of circular cross section comprised of titanium and operating at a frequency of about 20 kHz, the torsional wave speed is as follows:

$${c}_{t} = \sqrt{\frac{E / 2 ( 1 + v )}{ρ}} = \sqrt{\frac{1.1 \times {10}^{11} Pa / 2 ( 1 + 0.3 )}{4600 kg \text{/} {m}^{3}}} = 3032 m \text{/} s$$

Thus, the length of the ultrasonic probe should be an integer multiple of:

$$L = \frac{λ}{2} = \frac{c}{2 f} = \frac{3032 m \text{/} s}{2 ( 20 \text{,} 000 Hz )} = 0.0758 m = 7.58 cm \approx 3 in .$$

As a result, for the ultrasonic probe to operate in a pure torsional mode, the length of the ultrasonic probe should be an integer multiple of 7.58 cm (about 3 inches) for this particular case. Those skilled in the art will recognize that changes to other material properties can influence the operation in the torsional mode and these changes are within the spirit and scope of the present invention.

The present invention provides an apparatus and a method for an ultrasonic medical device operating in a torsional mode. An ultrasonic probe 15 of the ultrasonic medical device 11 undergoes a torsional vibration that produces a rotation and a counterrotation along the longitudinal axis of the ultrasonic probe 15. The torsional vibration of the ultrasonic probe 15 is projected in a forward and a reverse direction about a plurality of torsional nodes 50 of the ultrasonic probe 15. The present invention provides an ultrasonic medical device that is simple, user-friendly, time efficient, reliable and cost effective.

All patents, patent applications, and published references cited herein are hereby incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

1. An ultrasonic medical device comprising:
an ultrasonic probe comprising a proximal end, a distal end and a longitudinal axis therebetween;
a transducer creating a torsional vibration of the ultrasonic probe;
a coupling engaging the proximal end of the ultrasonic probe to a distal end of the transducer; and
an ultrasonic energy source engaged to the transducer that produces an ultrasonic energy.

2. The ultrasonic medical device of claim 1 wherein the torsional vibration of the ultrasonic probe causes a rotation and counterrotation along the longitudinal axis of the ultrasonic probe.

3. The ultrasonic medical device of claim 1 wherein the torsional vibration of the ultrasonic probe is propagated in a forward direction and a reverse direction about a plurality of torsional nodes along a portion of the longitudinal axis of the ultrasonic probe.

4. The ultrasonic medical device of claim 1 wherein a portion of the longitudinal axis of the ultrasonic probe comprises a radially asymmetric cross section.

5. The ultrasonic medical device of claim 4 wherein cavitation occurs around the portion of the longitudinal axis of the ultrasonic probe comprising the radially asymmetric cross section to ablate a biological material.

6. The ultrasonic medical device of claim 1 wherein the torsional vibration of the ultrasonic probe produces a plurality of torsional nodes and a plurality of torsional anti-nodes along a portion of the longitudinal axis of the ultrasonic probe.

7. The ultrasonic medical device of claim 1 wherein a length of the longitudinal axis of the ultrasonic probe comprises an approximately rectangular shaped cross section.

8. The ultrasonic medical device of claim 1 wherein a length of the longitudinal axis of the ultrasonic probe comprises a spline shape.

9. The ultrasonic medical device of claim 1 wherein a plurality of projections extend from an outer surface along a length of the ultrasonic probe.

10. The ultrasonic medical device of claim 1 wherein a length of the longitudinal axis of the ultrasonic probe has a cross sectional shape selected from the group consisting of elliptical, star shaped, rectangular, oval, triangular, trapezoidal, circular with a flat spot and square.

11. The ultrasonic medical device of claim 1 wherein the torsional vibration generates acoustic energy in a medium surrounding the ultrasonic probe.

12. The ultrasonic medical device of claim 1 wherein the ultrasonic energy source delivers ultrasonic energy in a frequency range from about 10 kHz to about 100 kHz.

13. The ultrasonic medical device of claim 1 wherein the ultrasonic energy source provides an electrical power to the transducer at a resonant frequency of the transducer by finding the resonant frequency of the transducer.

14. The ultrasonic medical device of claim 1 wherein the ultrasonic probe supports the torsional vibration when flexed.

15. The ultrasonic medical device of claim 1 wherein the ultrasonic probe has a flexibility allowing the ultrasonic probe to be deflected and articulated.

16. The ultrasonic medical device of claim 1 wherein the ultrasonic probe comprises a substantially uniform cross section from the proximal end of the ultrasonic probe to the distal end of the ultrasonic probe.

17. The ultrasonic medical device of claim 1 wherein the ultrasonic probe comprises a varying cross section from the proximal end of the ultrasonic probe to the distal end of the ultrasonic probe.

18. The ultrasonic medical device of claim 1 wherein the ultrasonic probe is disposable.

19. A medical device comprising:
an elongated probe comprising a proximal end, a distal end, and a longitudinal axis between the proximal end and the distal end wherein a portion of the longitudinal axis comprises a radially asymmetric cross section;
a transducer that converts electrical energy into mechanical energy, creating a torsional vibration along the longitudinal axis of the elongated probe;
a coupling engaging the proximal end of the elongated probe to a distal end of the transducer; and
an ultrasonic energy source engaged to the transducer that provides the electrical energy to the transducer,
wherein the torsional vibration along the elongated probe produces a plurality of torsional nodes and a plurality of torsional anti-nodes along a portion of the longitudinal axis of the elongated probe.

20. The medical device of claim 19 wherein the torsional vibration of the elongated probe produces a rotation and a counterrotation along the longitudinal axis of the elongated probe.

21. The medical device of claim 19 wherein the torsional vibration of the elongated probe is propagated in a forward direction and a reverse direction about the plurality of torsional nodes of the elongated probe.

22. The medical device of claim 19 wherein the torsional vibration generates acoustic energy in a medium surrounding the elongated probe.

23. The medical device of claim 19 wherein cavitation occurs over an active area of the elongated probe along the portion of the longitudinal axis comprising the radially asymmetric cross section.

24. The medical device of claim 19 wherein a length of the longitudinal axis of the elongated probe comprises a spline shape.

25. The medical device of claim 19 wherein a length of the longitudinal axis of the elongated probe has a cross sectional shape selected from the group consisting of elliptical, star shaped, rectangular, oval, triangular, trapezoidal, circular with a flat spot and square.

26. The medical device of claim 19 wherein a plurality of projections extend from an outer surface along a length of the elongated probe.

27. The medical device of claim 19 wherein the ultrasonic energy source delivers ultrasonic energy in a frequency range from about 10 kHz to about 100 kHz.

28. The medical device of claim 19 wherein the ultrasonic energy source provides an electrical power to the transducer at a resonant frequency of the transducer by finding the resonant frequency of the transducer.

29. The medical device of claim 19 wherein the elongated probe supports the torsional vibration when flexed.

30. The medical device of claim 19 wherein the elongated probe has a flexibility allowing the elongated probe to be deflected and articulated.

31. The medical device of claim 19 wherein the elongated probe comprises a substantially uniform cross section from the proximal end of the elongated probe to the distal end of the elongated probe.

32. The medical device of claim 19 wherein the elongated probe comprises a varying cross section from the proximal end of the elongated probe to the distal end of the elongated probe.

33. The medical device of claim 19 wherein the elongated probe has an approximately circular cross section.

34. A method of treating a biological material in a body with an ultrasonic medical device comprising:
providing the ultrasonic medical device comprising an ultrasonic probe having a proximal end, a distal end and a longitudinal axis therebetween wherein a portion of the longitudinal axis comprises a radially asymmetric cross section;
moving the ultrasonic probe to a treatment site of the biological material to place the ultrasonic probe in communication with the biological material; and
activating an ultrasonic energy source engaged to the ultrasonic probe to produce an ultrasonic energy that is converted into a torsional vibration of the ultrasonic probe.

35. The method of claim 34 further comprising creating the torsional vibration along the longitudinal axis of the ultrasonic probe with the ultrasonic energy source engaging a proximal end of a transducer and the ultrasonic probe engaging a distal end of the transducer.

36. The method of claim 34 further comprising generating acoustic energy in a medium surrounding the ultrasonic probe through the torsional vibration of the ultrasonic probe.

37. The method of claim 34 further comprising producing a plurality of torsional nodes and a plurality of torsional anti-nodes along a portion of the longitudinal axis of the ultrasonic probe by the torsional vibration of the ultrasonic probe.

38. The method of claim 34 wherein cavitation occurs around an active area of the ultrasonic probe comprising the portion of the longitudinal axis having the radially asymmetric cross section.

39. The method of claim 34 wherein a length of the longitudinal axis of the ultrasonic probe comprises a spline shape.

40. The method of claim 34 wherein a length of the longitudinal axis of the ultrasonic probe has a cross sectional shape selected from the group consisting of elliptical, star shaped, rectangular, oval, triangular, trapezoidal, circular with a flat spot and square.

41. The method of claim 34 wherein a plurality of projections extend from an outer surface along a length of the ultrasonic probe.

42. The method of claim 34 further comprising producing a rotation and counterrotation along the longitudinal axis of the ultrasonic probe by the torsional vibration of the ultrasonic probe.

43. The method of claim 34 further comprising projecting the torsional motion of the ultrasonic probe in a forward direction and a reverse direction about a plurality of torsional nodes of the ultrasonic probe.

44. The method of claim 34 further comprising sweeping the ultrasonic probe along the treatment site of the biological material.

45. The method of claim 34 further comprising moving the ultrasonic probe back and forth along the treatment site of the biological material.

46. The method of claim 34 further comprising rotating the ultrasonic probe along the treatment site of the biological material.

47. The method of claim 34 further comprising delivering ultrasonic energy in a frequency range from about 10 kHz to about 100 kHz by the ultrasonic energy source.

48. The method of claim 34 further comprising providing an electrical power to a transducer at a resonant frequency of the transducer of the ultrasonic medical device by the ultrasonic energy source determining the resonant frequency of the transducer.

49. The method of claim 34 further comprising providing the ultrasonic probe having a flexibility allowing the ultrasonic probe to be deflected and articulated.

50. The method of claim 34 further comprising providing the ultrasonic probe having a flexibility to support the torsional vibration when flexed.

51. The method of claim 34 further comprising providing the ultrasonic probe having a substantially uniform cross section from the proximal end of the ultrasonic probe to the distal end of the ultrasonic probe.

52. The method of claim 34 further comprising providing the ultrasonic probe having a varying cross section from the proximal end of the ultrasonic probe to the distal end of the ultrasonic probe.

53. A method of removing a biological material in a body comprising:
providing an ultrasonic medical device comprising an ultrasonic probe having a proximal end, a distal end that terminates in a probe tip and a longitudinal axis between the proximal end and the distal end;
moving the ultrasonic probe in the body and placing the ultrasonic probe in communication with the biological material; and
activating an ultrasonic energy source of the ultrasonic medical device to produce an electric signal that drives a transducer of the ultrasonic medical device to produce a torsional vibration of the ultrasonic probe, wherein the torsional vibration of the ultrasonic probe produces a plurality of torsional nodes and a plurality of torsional anti-nodes along a portion of the longitudinal axis of the ultrasonic probe.

54. The method of claim 53 wherein a portion of the longitudinal axis of the ultrasonic probe has a radially asymmetric cross section.

55. The method of claim 53 wherein the plurality of torsional anti-nodes are points of a maximum torsional vibration.

56. The method of claim 53 further comprising producing a rotation and counterrotation along the longitudinal axis of the ultrasonic probe by the torsional vibration of the ultrasonic probe.

57. The method of claim 53 further comprising projecting the torsional vibration of the ultrasonic probe in a forward direction and a reverse direction about the plurality of torsional nodes of the ultrasonic probe.

58. The method of claim 53 wherein cavitation occurs along an active area of the ultrasonic probe along a portion of the longitudinal axis comprising a radially asymmetric cross section.

59. The method of claim 53 wherein cavitation occurs at the probe tip.

60. The method of claim 53 wherein a length of the longitudinal axis of the ultrasonic probe comprises a spline shape.

61. The method of claim 53 wherein a length of the longitudinal axis of the ultrasonic probe comprises a cross sectional shape selected from the group consisting of elliptical, star shaped, rectangular, oval, triangular, trapezoidal, circular with a flat spot and square.

62. The method of claim 53 further comprising delivering ultrasonic energy in a frequency range from about 10 kHz to about 100 kHz by the ultrasonic energy source.

63. The method of claim 53 wherein the ultrasonic probe is for a single use on a single patient.

64. An ultrasonic probe comprising:
a proximal end;
a distal end that terminates in a probe tip; and
a longitudinal axis between the proximal end and the distal end, a portion of the longitudinal axis of the ultrasonic probe comprising a radially asymmetric cross section to support a torsional vibration.

65. The ultrasonic probe of claim 64 wherein the ultrasonic probe comprises a varying cross section from the proximal end of the ultrasonic probe to the distal end of the ultrasonic probe.

66. The ultrasonic probe of claim 64 wherein a cross section of the proximal end of the ultrasonic probe is approximately circular.

67. The ultrasonic probe of claim 64 wherein the radially asymmetric cross section comprises a spline shape.

68. The ultrasonic probe of claim 64 wherein the radially asymmetric cross section has a cross sectional shape selected from the group consisting of elliptical, star shaped, rectangular, oval, triangular, trapezoidal, circular with a flat spot and square.

69. The ultrasonic probe of claim 464 wherein the ultrasonic probe comprises a varying diameter from the proximal end of the ultrasonic probe to the distal end of the ultrasonic probe.

70. The ultrasonic probe of claim 64 wherein the ultrasonic probe has a flexibility allowing the ultrasonic probe to be deflected and articulated.

71. The method of claim 53 wherein the plurality of torsional nodes are points of a minimum torsional vibration.
